(19) United States
(12) Patent Application Publication
(10) Pub. No.: US 2008/0004533 A1
(43) Pub. Date: Jan. 3, 2008

(54) Optical imaging systems and methods

(75) Inventors: Floribertus P. M. Heukensfeldt Jansen, Niskayuna, NY (US); Stephen Johnson Lomnes, Philadelphia, PA (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/480,162

(22) Filed: Jun. 30, 2006

(51) Int. Cl. A61B 6/00

(52) U.S. Cl. 600/476

(57) ABSTRACT

Optical imaging systems and methods are disclosed. The optical imaging system includes an electronic imaging device configured to capture an image of a site and a projector. An optical imaging system includes a projector configured to project a visible representation of the captured image onto the site during surgery. Another optical imaging system includes an optical element capable of aligning an optical axis of the electronic imaging device and the projector on a same optical axis. Also disclosed are methods for displaying an image on to a site. A method includes capturing a fluorescent image of a surgical site; and projecting a visible representation of the captured image onto the surgical site during surgery. Another method includes projecting a visible representation of a captured image of a site onto the site along a same optical axis along which the image is captured.

### BACKGROUND

The invention includes embodiments that may relate to optical imaging. Particularly, the invention includes embodiments that may relate to medical imaging systems and methods.

Although some optical imaging methods may be used to visualize and display a living tissue of interest, such as a surgical site, the methods indirectly visualize the surgical site by looking at a computer monitor of the surgical site, instead of directly at the surgical site. Thus, a surgeon operates by looking at a monitor instead of at the surgical site. Furthermore, when operating, the surgeon may not know the exact location or size of the operation site, such as a tumor. Consequently, a need still exists for optical imaging methods and systems.

### BRIEF DESCRIPTION

The embodiments of the invention will be set forth and apparent from the description that follows, as well as will be learned by practice of the embodiments of the invention. Additional aspects will be realized and attained by the methods and systems particularly pointed out in the written description and claims hereof, as well as from the appended drawings.

An aspect of the invention includes an optical imaging system. The optical imaging system includes an electronic imaging device and a projector. The electronic imaging device is configured to capture an image of a surgical site. The projector is configured to project a visible representation of the captured image onto the site during surgery.

Another aspect of the invention includes a method for displaying an image onto a site. The method includes illuminating a surgical site that is fluorescent or labeled with a fluorescent agent with an excitation light source that provides one or more wavelengths to excite the fluorescent agent or fluorescent site; capturing an image that is infrared fluorescent of the surgical site; and projecting a visible representation of the captured image onto the surgical site during surgery.

Another aspect of the invention includes a method for displaying an image onto a site. The method includes providing an electronic imaging device, a projector, and an optical element. The electronic imaging device is configured to capture an image of a surgical site. The projector is configured to project a visible representation of the captured image. The optical element is capable of aligning an optical axis of the electronic imaging device and the projector on a same optical axis.

Another aspect of the invention includes an optical imaging system. The optical imaging system includes an electronic imaging device, projector, and an optical element. The electronic imaging device is configured to capture an image of a surgical site that is fluorescent. The projector is configured to project a visible representation of the captured image onto the site. The optical element is capable of aligning an optical axis of the electronic imaging device and the projector on a same optical axis.

The foregoing general description and the following detailed description are exemplary and are intended to provide further explanation of the invention claimed.

The accompanying figures, which are incorporated in and constitute part of this specification, are included to illustrate and provide a further understanding of the method and system of the invention. Together with the description, the drawings explain the principles of the invention.

### BRIEF DESCRIPTION OF THE DRAWINGS

FIG. 1 is a schematic representation of an optical imaging system in accordance with the invention;

FIG. 2 is a schematic representation of another optical imaging system in accordance with the invention.

FIG. 3 is a flow chart of a method of displaying infrared fluorescent data in accordance with the invention; and

FIG. 4 is another flow chart of a method of displaying infrared fluorescent data in accordance with the invention.

These and other features, aspects, and embodiments of the invention will become better understood when the following detailed description is read with reference to the accompanying drawings in which like characters represent like parts throughout the drawings.

### DETAILED DESCRIPTION

Exemplary embodiments of the invention are illustrated in the accompanying figures and examples. Referring to the drawings in general, the illustrations describe a particular embodiment of the invention and do not limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of parts and/or variables are permissible only if such combinations result in stable operable systems.

The methods and systems presented herein may achieve one or more of the following: projecting a visible representation of a captured image of a surgical site onto the surgical site during surgery; and projecting a visible representation of a captured image of a surgical site along a same optical axis along which the image is captured. Particularly disclosed is a system and method of operating while looking at a surgical site directly instead of a monitor of the surgical site.

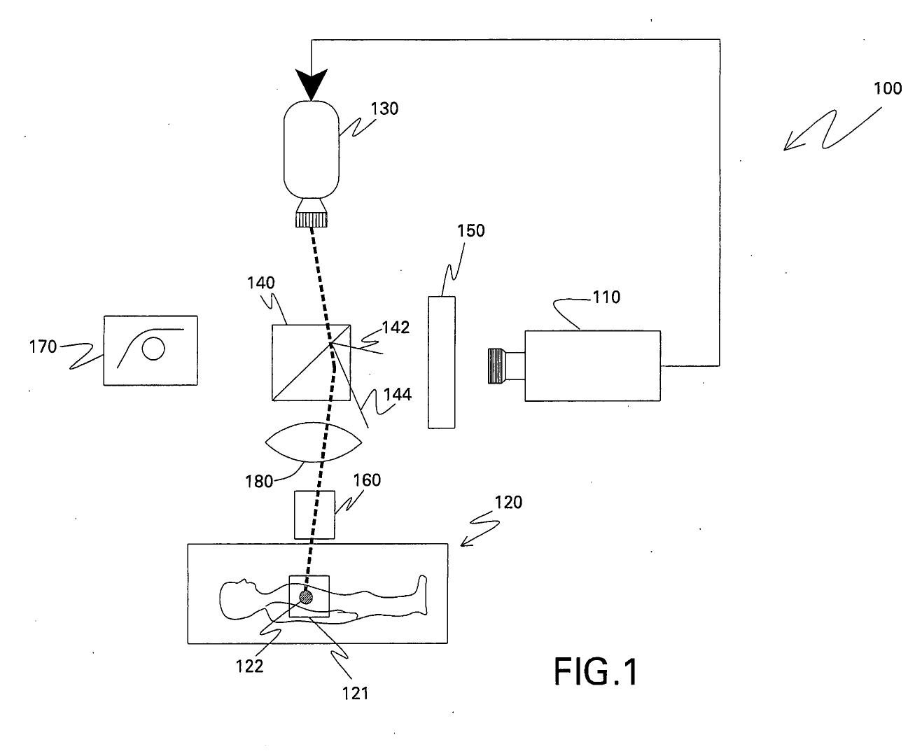

An embodiment of an optical imaging system 100 is shown in FIG. 1. The optical imaging system includes an electronic imaging device 110 and a projector 130. The electronic imaging device is configured to capture an image of a site 121, such as, but not limited to, a surgical site of a patient 120. The projector is configured to project a visible representation of the captured image onto the site. In one embodiment, the image is a surgical site and the projector is configured to project a visible representation of the captured image onto the surgical site during surgery.

During surgery is not limited by how long the image is projected during surgery nor by the time when the image is projected during surgery, and also not limited by the time interval between when the image is captured and projected. During surgery includes a time interval from almost instantaneously up to about 60 minutes from when the image is captured and projected. In one embodiment, during surgery includes a range of 1-30 seconds, 30-60 seconds, 1-5 minutes, 5-10 minutes, 10-15 minutes, 15-20 minutes, 20-25 minutes, 25-30 minutes, and 30-35 minutes from when the image is captured and projected. During surgery may also include longer time intervals between when the image is captured and projected, such as when the surgery may be 6 hours, 12 hours, etc., as long as the image is projected at some point during the surgery. During surgery is not limited by how long the image is projected during surgery and includes any amount of time during surgery. In some embodiments, during surgery includes projecting an image during the surgery for 1-10% 20%-30%, 30%-40%, 40%-50%, 60%-70%, or 70%-80% of the surgical time period. In a particular embodiment, during surgery includes projecting an image during the surgery for 1-10% of the surgical time period. During surgery is also not limited by the time when the image is projected during surgery, and includes projecting an image during the surgery at any time, such as the beginning, middle, or end of the surgery, and includes continuous or intermittent projections during surgery.

### Fluorescent Agent or Site

The site 121 may include any areas of a subject's body, such as a tumor, lesion 122, or other areas of interest, as shown in FIG. 1. For example, the site 121 maybe a surgical site for an open chest bypass. Examples of subjects include mammals, such as people. Other suitable mammals include, but are not limited to, rats, pigs, etc. Examples of subjects also include other animals besides mammals.

Prior to or during the imaging procedure, the surgical site may be inherently fluorescent, labeled with a fluorescent agent, or bioluminescent, either individually or in a combination of two or more. Examples of inherently fluorescent sites include blood vessels, nervous system, lymph node, and digestive tract. Inherently fluorescent sites may also be labeled with a fluorescent agent to increase fluorescence at a predetermined level based on desired level of fluorescence. The system and method is not limited by the type of fluorescent agent nor by how the fluorescent agent is provided. The system and method is also not limited by the type of surgery and includes various medical interventions.

### Illumination Source

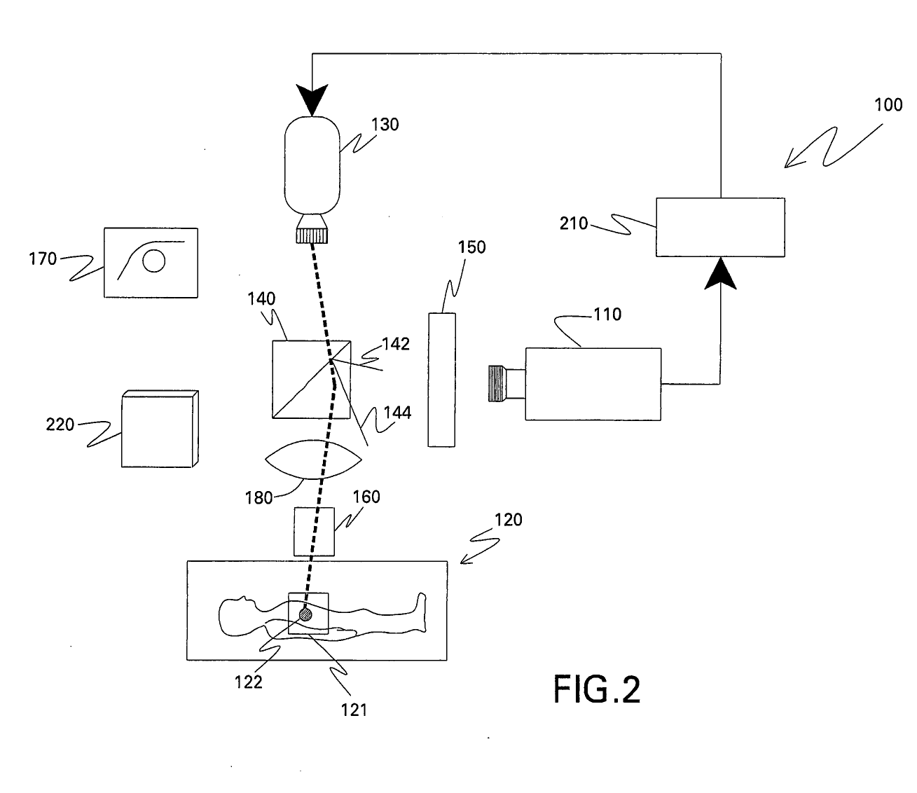

The optical imaging system may further include one or more illumination sources 170, as shown in FIG. 2, to excite the fluorescence. The illumination source provides one or more wavelengths to excite the fluorescent agent or the inherently fluorescent site. The illumination source may be, for example, a laser diode such as a 771 nm, 250 mW laser diode system, which may be obtained from Laser Components of Santa Rosa, Calif. Other single wavelength, narrowband, or broadband light sources may be used, provided they do not interfere with the visible light image captured by the electronic imaging device or the emission wavelength of the fluorescent agent. In one embodiment, the excitation wavelength includes wavelengths from about 600 nm to about 1300 nm. This includes some portions of the visible spectrum, such as orange and red in a range from about 600-700 nm, and the shorter wavelengths of the near-infrared bands. In another embodiment, the excitation wavelength includes “near infrared” wavelengths, generally from 700 nm to 3000 nm.

Other non-limiting examples of illumination sources may include a light source such as a laser, laser diode, light emitting diode (LED), or a lamp (e.g., halogen lamp, incandescent lamp, arc lamp, high intensity discharge lamp etc.), either individually or in a combination of two or more, to provide illumination as desired. In one embodiment, the illumination source is an ultra-short laser pulse or a sequence of such pulses. Alternatively, in certain embodiments, multiple illumination sources deliver the light via optical fiber or fiber optic bundles to form an array of localized illumination spots that may be illuminated simultaneously or in a sequence. The illumination source can be operated in continuous wave (CW) mode, intensity modulated wave mode, or pulsed wave mode. The illumination sources also includes bioluminescence, either individually or in a combination of two or more illumination sources, discussed above to provide illumination as desired.

When excitation photons reach the fluorescent agent in the object of interest, e.g., a lesion, localized in a volume of biological tissue, the fluorescence may re-emit optical radiation at a longer wavelength and acts as an omnidirectional optical source. As will be appreciated by one skilled in the art, the emitted light from fluorescence may include ballistic photons, snake-like photons, and/or diffused photons.

Furthermore, suitable optical coupling and lenses may be provided to direct each of the illumination light sources at an area of interest within a surgical site or multiple sites.

### Electronic Imaging Device

The optical imaging system 100 may further include one or more electronic imaging devices 110, as shown in FIGS. 1 and 2. The electronic imaging device is configured to or capable of capturing one or more images of the surgical site. A variety of electronic imaging device may be used to capture an image of the surgical site, such as, but not limited to, a single optical detector (e.g., a photodetector), a photomultiplier tube (PMT), a photodiode, a photon counter, an image intensifier, arrays of photosensitive elements (array of photodetectors), full field optical detectors such as charge-coupled device (CCD) cameras, a complementary metal oxide semiconductor (CMOS) device, avalanche photodiodes, a photo-refractive interferometer, and/or a full field speckle interferometer. Particular examples electronic imaging devices include a charge-coupled device, and x-ray, video, gamma, ultrasound, MRI, PET, and cameras, either individually or in a combination of two or more. The electronic imaging device is capable of recording information about the surgical field that may or may not be readily observed with the naked eye, this may include but is not limited to absorption, scatter, fluorescence, multi-spectral optical signature, radioisotope emission, or data characteristic detectable by MRI, Xray or other imaging platforms. Although particular embodiments described herein relate to fluorescent imaging, the invention is not intended to be limited to such. For example, other scanning platforms are capable of detecting barely visible or non-visible (to the naked eye) image information and converting or enhancing the information into an optical signal that would be useful and applicable to embodiments of the optical imaging system described herein.

In a particular embodiment, the electronic imaging device includes a camera with a filter 150, as shown in FIG. 1. The camera may be an infrared (IR) camera. One or more optical filters, as part of a camera, or separately, may be provided to preferentially transmit particular type or wavelength of light. In one embodiment, the filter may transmit IR light while in another embodiment, the filter may transmit visible light. In one embodiment, the filter selectively transmits the fluorescence wavelength to allow regions of the site that are not visible to the naked eye to be captured by a camera. In certain embodiments, the system may also include an amplifier for amplifying the captured image or signal. Furthermore, the electronic imaging device may capture multiple images of one or more surgical sites, sequentially or simultaneously.

In one embodiment, multiple electronic imaging devices may capture multiple images of one or more surgical sites, sequentially or simultaneously. A particular embodiment includes at least two electric imaging devices, wherein an electric imaging device configured to visualize the surgical site and another electric imaging device is configured to visualize the projected image. In another embodiment, a camera may alternate its sensitivity between the fluorescent emitted light and the projected light, such as by a tunable filter.

### Projector

The optical imaging system 100 may further include one or more projectors 130, as shown in FIGS. 1 and 2. The projector is configured to project a visible representation of the captured image onto the surgical site. The projector may project visible representations of multiple captured images onto more than one surgical site. A variety of projectors may be used to project a visible representation of the captured image onto the surgical site, such as but not limited to, a liquid crystal display projector and liquid crystal light valve. Other reflective or transmissive projections can be used with appropriate modifications, such as a laser with steerable mirrors, an array of light emitting elements, such as LEDs or laser diodes. The projector displays a representation of the captured image; the representation of the image may be transformed in various ways to make the image visible. The image may be transformed in various ways such as applying a color scale to the intensity of fluorescent intensity measured. For example, in a 16 bit CCD camera, a value of 65,535 may be converted to an RGB value of [0 1 0], and projected as a maximum intensity of green, while a 32,764 value is converted to an RGB value of [0 0.5 0], and projected as green at a half a maximum intensity. More sophisticated transformations may be employed to improve the utility of the imaging system, such as non-linear transformation, and the application of various color maps.

### Optical Element

In another embodiment, the optical imaging system 100 includes one or more optical elements 140, as shown in FIGS. 1 and 2, whether the image is or is not projected onto the site during surgery. The optical element is capable of aligning an optical axis of the electronic imaging device and the projector on a same optical axis 160.

On a same optical axis also includes on about a same optical axis. Approximating language, as used herein throughout the specification and claims, may be applied to modify any quantitative or qualitative representation that could permissibly vary without resulting in a change in the basic function to which it is related. Accordingly, a value modified by a term such as “about” is not to be limited to the precise value specified, and may include values that differ from the specified value. In at least some instances, the approximating language may correspond to the precision of an instrument for measuring the value. Similarly, “same or about same” may be used in combination with a term, and may include an insubstantial number or trace amount of a difference or modification while still being considered same or about same.

When the optical axes are exactly aligned, registration of the captured fluorescent image and the projected imaged is ensured regardless of surface variation. When the axes are approximately aligned, calibration of the projector and the electronic imaging device can ensure good registration at one object distance, while alignment variation may occur at other object distances. Accurate alignment may exist in some embodiments; however, range of differences may be allowed while still being considered on a same optical axis. For example, the optical axis of the electronic imaging device and the projector may have a difference of up to 5 mm.

In one embodiment, the optical element includes a beam splitter. The beam splitter is configured to separate transmission 142 and reflection 144 over different wavelength bands or to produce a specific reflectance and transmission at a wavelength or a wavelength band, as shown in FIGS. 1 and 2. For example, the beam splitter may be adjusted in two ways, such as reflecting visible light while transmitting IR light or reflecting IR. The beam splitter may align an optical axis of the electronic imaging device and the projector on a same optical axis by turning the light of a signal at a fixed angle, such as 90 degrees. The optical axis may also be aligned by mechanically moving the camera and the projector or moving the beam splitter.

Examples of beam splitter include, but are not limited to, a dichroic filters, partially reflective mirrors, cube beam-splitters, and polarizing beam splitters, either individually or in a combination of two or more.

Multiple beam splitters may be used to align the optical axis of the electronic imaging device and the projector on a same optical axis. The multiple beam splitters can be disposed at a predetermined angle and or location relative to the projector and the electronic imaging device and to each other. The beam splitter may include glass elements, such as generally planar glass elements. The glass elements can be coated to provide reflection of a desired percent. Particularly, the beam splitters may have a coating adapted so that the deflected portion directed by the multiple beam splitters will have a similar relative intensity. The glass elements further may have varying thickness, such as, but not limited to, in the range of about 0.25 mm to about 1.0 mm. Examples of glass elements include sawn Pyrex® wafers having a thickness of about 0.25 mm, although other dimensions may be suitable, as will become apparent to those skilled in the art.

The beam splitter may be constructed of a glass plate having a pair of generally planar surfaces. For examples, glass slides commonly used in a chemical laboratory have been shown to be suitable. To achieve more precise control over the optical properties of the beam splitter, the beam splitter can include an optical coating on each of the surfaces, respectively. Furthermore, the coating on each of the beam splitters may be variable to direct light as predetermined. The coating may provide the beam splitter with a predetermined reflective, refractive and other optical properties. The beam splitter may have a reflection of varying ranges.

Furthermore, the optical imaging system 100 may also include one or more lenses 180 to align optical axis of the electronic imaging device and the projector on a same optical axis. Examples of lens include an objective lens.

### Geometric Calibrator and Mirror

Referring to FIG. 2, the optical imaging system 100 may further include one or more geometric calibrators 210 and one or more mirrors 220. The geometric calibrator and mirror are configured to or capable of further aligning the optical axis of the electronic imaging device and the projector on a same optical axis. The geometric calibrator, mirror, or mechanical movement of the camera or projector, either individually or in a combination of two or more may be used to fine tune the alignment.

In one embodiment, the geometric calibrator may be a computer, data or image processing system. When aligned on a same optical axis, an image primarily composed of light in the NIR region and one in the visible region along the same optical axis may be less than optimal because of wavelength dependent variation in the optical components. A calibration process may be performed manually or automatically. Manual calibration of this system may involve applying a transformation matrix operation (scale, stretch, rotate, and translate) to the image data prior to projection. The terms within the transformation matrix may be modified until the misalignment between a test image and the projected image is minimized.

In another embodiment, whether aligned or not aligned on a same optical axis, the calibration of this system may be completed automatically by placing a test sample (e.g. a uniformly colored subject with a pattern of fluorescent material embedded in or on the test sample), exciting the sample with the excitation source, capturing the fluorescent image, applying an initial geometric transformation, converting the image to color representation (e.g. RGB), projecting the color image back onto the sample, capturing an additional image using a camera that is sensitive to the visible spectrum, defining an objective function that represents the differences between the fluorescent image and the image of the projected visible image, and minimizing this objective function by modifying the terms in the transformational matrix. The minimization can occur by any of a set of well-known minimization routines. The objective function can be defined in a number of mutual information routines, such a cross-correlation etc. As will be appreciated by one skilled in the art, the image processing system may be implemented in various forms of hardware, software, firmware, special purpose processors, or a combination thereof. In one embodiment, the geometric calibrator (data processing system) may be implemented in software as an application program tangibly embodied on a program storage device. The application program may be uploaded to, and executed by, a machine having any suitable architecture. Particularly, the machine is implemented on a computer platform having hardware such as one or more central processing units (CPU), a random access memory (RAM), a read only memory (ROM) and input/output (I/O) interface(s) such as a keyboard, a cursor control device (e.g., a mouse) and a display device (monitor). The computer platform also includes an operating system and microinstruction code. The various processes and functions described herein may either be part of the microinstruction code or part of the application program (or a combination thereof), which is executed via the operating system. In addition, various other peripheral devices may be connected to the computer platform such as an additional data storage device and a printing device.

One or more mirrors may also be included to align the optical axis of the electronic imaging device and the projector on a same optical axis

The optical imaging system includes multiple optical elements, as well as geometric calibrators and mirrors to align the optical axis of the electronic imaging device and the projector on a same optical axis, either sequentially or simultaneously. When sequentially, the system or method is not restricted by the order or number of the optical elements, geometric calibrators, and mirrors.

The optical imaging system includes multiple electronic imaging devices or multiple projectors, that are or are not aligned on the same optical axis and may or may not project a captured image onto the site during surgery. In a particular embodiment of the optical imaging system, a projector is configured to project a visible representation of the captured image onto the site during surgery and an optical element is capable of aligning an optical axis of the electronic imaging device and the projector on a same optical axis.

The system also includes the use of multiple illumination sources to illuminate a plurality of sites with lights of distinguishable colors. Differing colors of light may be generated by fluorescence by adjusting or controlling the fluorescent agent as well as the environmental factors. For example, environmental factors such as pH, concentration of ions like sodium, zinc, Ca, and Mg, and temperature, either individually or in combinations, may affect the emission wavelength and hence the color generated. In fact, environmental factors such as pH and concentrations of ions can affect luminescence even to the point of whether there will be or will not be luminescence. Although the environment of the surgical site may also affect the emission spectrum, one of ordinary skill in the art can make adjustments to correct for such changes in the spectrum. There are existing publications demonstrating a shift in emission wavelength as a function of various environmental factors.

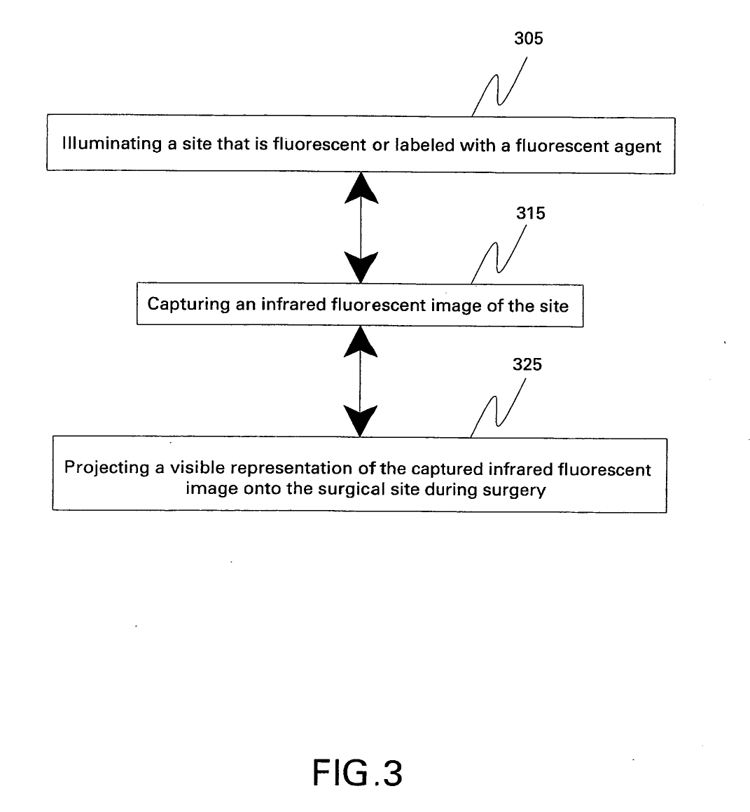

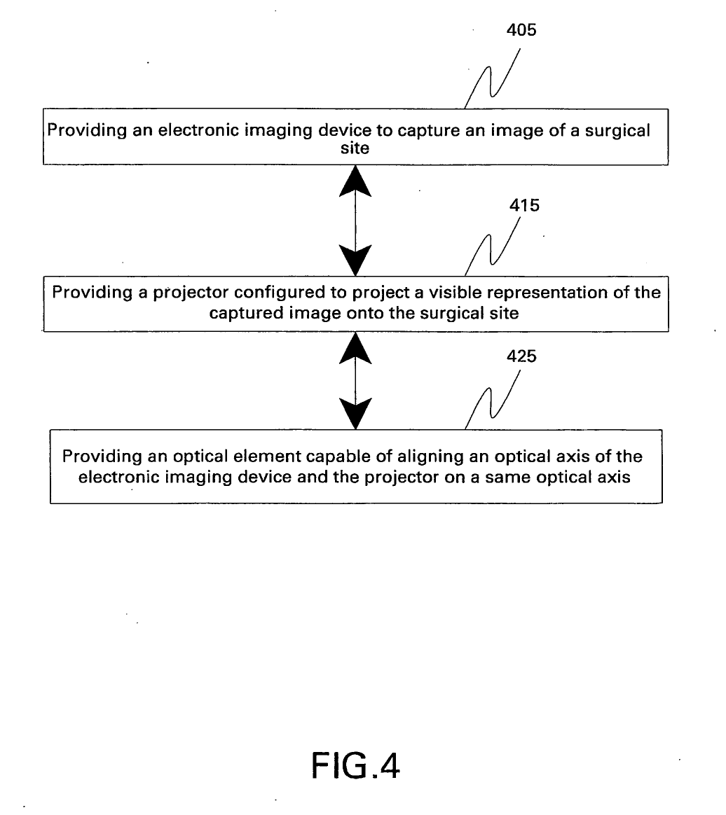

Referring to FIG. 3 and FIG. 4, methods for displaying images onto a site are described. In one embodiment, as shown in FIG. 3, the method includes, Step 305, of illuminating a surgical site that is fluorescent or labeled with a fluorescent agent with an excitation light source that provides one or more wavelengths to excite the fluorescent agent or fluorescent site. Step 315 includes capturing an infrared fluorescent image of the surgical site. Step 325 includes projecting a visible representation of the captured image onto the surgical site during the surgery.

The method is not limited by how and when image is captured and projected. The image may be captured and projected simultaneously or sequentially, with varying time interval. In one embodiment, the infrared fluorescent image of the site is captured with an electronic imaging device, such as but not limited, to those described above. A visible representation of the captured infrared fluorescent image may be projected with a projector, such as but not limited, to those described above.

The method may further include aligning the optical axis of the electronic imaging device and the projector on a same optical axis. A particular embodiment of the method includes aligning the optical axis of the electronic imaging device and the projector on a same optical axis with an optical element such as a beam splitter. The optical axis of the electronic imaging device and the projector may also be further aligned on a same optical axis with a geometric calibrator or a mirror or both, in either combination or sequence.

The method also further includes enhancing the projected visible representation of the captured image. The projected visible representation of the captured image may also be enhanced by contrasting color, contrasting intensity, intensity modulation, contrasting pattern, either individually or in a combination of two or more. The method also includes allowing for the user to manually select a color representation, intensity, and frequency of providing the projected image to the user. For example the operator may choose a specific color map through selection in the user interface, and modify any of the contrast settings for suitable comfort. Additionally, the user may switch on or off the projection feature through the user interface, footswitch, voice or other input device to the system. The intensity may be modulated by changing the intensity of the illumination source in the projector, fluorescence excitation source, variable attenuation in any of the optical components, or in the digital representation of the fluorescent data in the computer, or by switching the projected image on or off in a particular time varying manner such that its appearance to a user is more or less intense.

As previously discussed, there may be a plurality of surgical sites or lesions of interest and the method is not restricted by the combination or sequence of the multiple images that are projected. In one embodiment, the method includes illuminating a plurality of sites that are fluorescent or labeled with a fluorescent agent with an excitation light source that provides one or more wavelengths to excite the fluorescent agent or fluorescent site. A plurality of infrared fluorescent images of the plurality of sites may be captured with an IR camera, and visible representations of the plurality of captured infrared fluorescent images may be projected onto the site with an LCD projector. Each of the images may be captured and projected by different mode of capture or electronic imaging device and projectors.

Referring to FIG. 4, another method for displaying an images onto a site is described. Step 405 includes providing an electronic imaging device configured to capture an image of a surgical site. Step 415 includes providing a projector configured to project a visible representation of the captured image onto the surgical site; and Step 425 includes providing an optical element capable of aligning an optical axis of the electronic imaging device and the projector on a same optical axis.

The method is not limited by how and when image is captured and projected. The electronic imaging device, projector, and optical element may be provided simultaneously or sequentially, and the kind of electronic imaging device, projector, and optical element provide may also vary.

The method may further include projecting a visible representation of the captured image onto the site during surgery.

The systems and methods described above are not limited by the type of fluorescent agent nor by how the fluorescent agent is provided. In embodiments using a fluorescent agent, the fluorescent agent may be delivered orally, topically, parenterally, by inhalation spray, rectally, subcutaneous injections, intravenous, intramuscular, intrasternal injection or infusion techniques, either individually or in a combination of two or more. For example, the fluorescent agent may be a fluorescent dye injected into the subject by an intravenous injection. The fluorescent agent may be sprayed onto the subject.

In one embodiment, the fluorescent agent accumulates at the surgical site or binds to the object of interest, such as the tumor or lesion. In one embodiment, accumulating includes a higher concentration of fluorescent agent at the surgical site compared to another portion of the subject. In a particular embodiment, accumulating includes a portion of the surgical site having at least 20% more fluorescent agent compared to another portion of the subject. In a more particular embodiment, accumulating includes a portion of the surgical site having at least 50% more fluorescent agent compared to another portion of the subject. In another particular embodiment, accumulating includes a portion of the surgical site having at least 70% more fluorescent agent compared to another portion of the subject. In other embodiments, a portion of the surgical site has 20%-30%, 30%-40%, 40%-50%, 60%-70%, or 70%-80% more fluorescent agent compared to another portion of the subject.

Examples of fluorescent agents include, but are not limited to, an antibody, an antibody fragment, or a low-molecular-weight ligand, rhodamine, Indocyanine green (ICG), a member of the Cy family of dyes, the IR-78 dye, peptide, cyanine, squarilium, croconium, merocyanine or oxonol dye molecule, lanthanide metal chelate, quantum dot, metal nano-cluster, fluorescein, and methylene blue, either individually or in a combination of two or more. The fluorescent agent may be one or more derivatives of the above or any other fluorophore that emits light in the near infrared region (NIR) region. The fluorescent agent may be soluble in blood. In one embodiment, the fluorescent agent will absorb and emit light in the “transparency window” of biological tissue, which is between 700-900 nm where absorption of light in tissue is minimized. In other applications, it may be useful to operate anywhere in the range from 400-2000 nm for use with light emitting agents that operate in these wavelengths.

In a particular embodiment, the fluorescent agent includes an antibody. The term “antibody” as used herein includes antibodies obtained from both polyclonal and monoclonal preparations, as well as hybrid (chimeric) antibody molecules; F(ab′)2 and F(ab) fragments; Fv molecules (noncovalent heterodimers); single-chain Fv molecules (sFv); dimeric and trimeric antibody fragment constructs; humanized antibody molecules; and any functional fragments obtained from such molecules, wherein such fragments retain specific-binding properties of the parent antibody molecule. The antibody may be a whole immunoglobulin of any class; e.g., IgG, IgM, IgA, IgD, IgE, chimeric or hybrid antibodies with dual or multiple antigen or epitope specificities. It can be a polyclonal antibody, particularly a humanized or an affinity-purified antibody from a human. It can be an antibody from an appropriate animal; e.g., a primate, goat, rabbit, mouse, or the like. If a paratope region is obtained from a non-human species, the target may be humanized to reduce immunogenicity of the non-human antibodies, for use in human diagnostic or therapeutic applications. Such a humanized antibody or fragment thereof is also termed “chimeric.” For example, a chimeric antibody includes non-human (such as murine) variable regions and human constant regions. A chimeric antibody fragment can includes a variable binding sequence or complementarity-determining regions (“CDR”) derived from a non-human antibody within a human variable region framework domain. Monoclonal antibodies are also suitable because of their high specificities. Useful antibody fragments include F(ab′)₂, F(ab)₂, Fab′, Fab, Fv, and the like including hybrid fragments. Particular fragments are Fab′, F(ab′)₂, Fab, and F(ab)₂. Also useful are any subfragments retaining the hypervariable, antigen-binding region of an immunoglobulin and having a size similar to or smaller than a Fab′ fragment. An antibody fragment can include genetically engineered and/or recombinant proteins, whether single-chain or multiple-chain, which incorporate an antigen-binding site and otherwise function in vivo as immobilized target-binding moieties in substantially the same way as natural immunoglobulin fragments. The fragments may also be produced by genetic engineering.

Mixtures of antibodies and immunoglobulin classes can be used, as can hybrid antibodies. Multispecific, including bispecific and hybrid, antibodies and antibody fragments are sometimes desirable for detecting and treating lesions and include at least two different substantially monospecific antibodies or antibody fragments, wherein at least two of the antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to anti-tumor marker hybrids.

It will be apparent to those skilled in the art that various modifications and variations can be made in the method and system of the present invention without departing from the spirit or scope of the invention. Thus, it is intended that the present invention include modifications and variations that are within the scope of the appended claims and their equivalents.

While the invention has been described in detail in connection with only a limited number of aspects, it should be understood that the invention is not limited to such disclosed aspects. Rather, the invention can be modified to incorporate any number of variations, alterations, substitutions or equivalent arrangements not heretofore described, but which are commensurate with the scope of the claims. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. An optical imaging system comprising:
an electronic imaging device configured to capture an image of a surgical site; and
a projector configured to project a visible representation of the captured image onto the surgical site during surgery.

2. The optical imaging system of claim 1, wherein the surgical site is selected from a group consisting of tumor, lesion, digestive tract, lymph node, and nervous system.

3. The optical imaging system of claim 1, wherein the surgical site is inherently fluorescent, labeled with a fluorescent agent, or bioluminescent.

4. The optical imaging system of claim 3, further comprising an excitation light source that provides one or more wavelengths to excite the fluorescent agent or fluorescent site.

5. The optical imaging system of claim 4, further comprising a filter that selectively transmits a fluorescence wavelength.

6. The optical imaging system of claim 1, further comprising an optical element capable of aligning an optical axis of the electronic imaging device and the projector on a same optical axis.

7. The optical imaging system of claim 6, wherein the optical element comprises a beam splitter.

8. The optical imaging system of claim 1, wherein the electronic imaging device preferentially detects infrared light.

9. The optical imaging system of claim 1, wherein the electronic imaging device further comprises a filter, wherein the filter preferentially transmits infrared light.

10. The optical imaging system of claim 1, wherein the electronic imaging device comprises at least a member selected from a group consisting of charge coupled device, video camera, x-ray, video, gamma, MRI, and PET cameras.

11. The optical imaging system of claim 1, further comprising a geometric calibrator or mirror; and
wherein the geometric calibrator or mirror are capable of aligning an optical axis of the electronic imaging device and the projector on a same optical axis.

12. The optical imaging system of claim 1, wherein the surgical site comprises a plurality of surgical sites that are inherently fluorescent, labeled with a fluorescent agent, or bioluminescent.

13. The optical imaging system of claim 12, wherein the electronic imaging device is configured to capture a plurality of images of the plurality of surgical sites that are different from each other; and
wherein the projector is configured to project visible representations of the plurality of the captured images onto the surgical site.

14. The optical imaging system of claim 1, wherein the electronic imaging device is alternately sensitive to fluorescent and visible light.

15. The optical imaging system of claim 1, wherein the electronic imaging device comprises at least two electric imaging devices, wherein an electric imaging device is configured to visualize the surgical site and another electric imaging device is configured to visualize the projected image.

16. The optical imaging system of claim 1, wherein the electronic imaging device is configured to record information about the surgical field and wherein the information comprises one of absorption, scatter, fluorescence, multi-spectral optical signature, radioisotope emission, and data detectable by MRI, Xray and medical imaging platforms and wherein the recorded information is converted to an optical signal for use in projecting onto the surgical site.

17. A method for displaying an image onto a site, comprising:
(i) illuminating a surgical site that is fluorescent or labeled with a fluorescent agent with an excitation light source that provides one or more wavelengths to excite the fluorescent agent or fluorescent surgical site;
(ii) capturing a fluorescent image of the surgical site; and
(iii) projecting a visible representation of the captured fluorescent image onto the surgical site during surgery.

18. The method of claim 17, wherein capturing the fluorescent image of the surgical site comprises capturing the fluorescent image with an electronic imaging device; and projecting a visible representation of the captured fluorescent image onto the surgical site with a projector.

19. The method of claim 18, further comprising aligning an optical axis of the electronic imaging device and the projector on a same optical axis with an optical element.

20. The method of claim 19, further comprising aligning the optical axis of the electronic imaging device and the projector on a same optical axis with a geometric calibrator or mirror.

21. The method of claim 17, wherein the electronic imaging device is alternately sensitive to fluorescent and visible light.

22. The method of claim 17, wherein the fluorescent image is captured by an electronic imaging device, and wherein the electronic imaging device comprises at least a member selected from a group consisting of charge coupled device, video camera, x-ray, video, gamma, MRI, and PET cameras.

23. The method of claim 17, further comprising enhancing the projected visible representation of the captured image.

24. The method of claim 23, wherein enhancing the projected visible representation of the captured image comprises contrasting color, contrasting intensity, or contrasting pattern.

25. The method of claim 17, wherein projecting a visible representation of the captured fluorescent image onto the surgical site during surgery comprises a time interval between when the image is captured and projected.

26. The method of claim 17, wherein the surgical site comprises a plurality of fluorescent regions.

27. The method of claim 17, further comprising illuminating a plurality of the surgical sites that are fluorescent or labeled with a fluorescent agent with an excitation light source that provides one or more wavelengths to excite the fluorescent agent or fluorescent surgical site.

28. The method of claim 17, further comprising capturing a plurality of fluorescent images of the of surgical sites.

29. The method of claim 17, further comprising projecting a plurality of visible representations of the captured fluorescent images onto the surgical site during surgery.

30. The method of claim 18, wherein the electronic imaging device comprises at least two electronic imaging devices, wherein an electronic imaging device is configured to visualize the surgical site and another electronic imaging device is configured to visualize the projected image.

31. The optical imaging system of claim 17, wherein the electronic imaging device is configured to record information about the surgical field and wherein the information comprises one of absorption, scatter, fluorescence, hyperspectral optical signature, radioisotope emission, and data detectable by MRI, Xray and medical imaging platforms and wherein the recorded information is converted to an optical signal for use in projecting onto the surgical site.

32. A method for displaying an image onto a site, comprising:
providing an electronic imaging device configured to capture an image of a surgical site;
providing a projector configured to project a visible representation of the captured image onto the surgical site; and
providing an optical element capable of aligning an optical axis of the electronic imaging device and the projector on a same optical axis.

33. The method of claim 32, further comprising projecting a visible representation of the captured image onto the surgical site during surgery.

34. An optical imaging system comprising:
an electronic imaging device configured to capture an image of a surgical site;
a projector configured to project a visible representation of the captured image onto the surgical site; and
an optical element capable of aligning an optical axis of the electronic imaging device and the projector on a same optical axis.

35. The optical imaging system of claim 34, wherein the surgical site is inherently fluorescent or labeled with a fluorescent agent.

36. The optical imaging system of claim 34, wherein the projector is configured to project a visible representation of the captured image onto the surgical site during surgery.

37. The optical imaging system of claim 34, wherein the electronic imaging device comprises at least a member selected from a group consisting of charge coupled device, video camera, x-ray, video, gamma, MRI, and PET cameras.

38. The optical imaging system of claim 34, wherein the electronic imaging device comprises at least two electronic imaging devices, wherein an electronic imaging device is configured to visualize the surgical site and another electronic imaging device is configured to visualize the projected image.
